(19) United States
(12) Patent Application Publication
(10) Pub. No.: US 2012/0165697 A1
(43) Pub. Date: Jun. 28, 2012

(54) SAMPLE COLLECTING DEVICE

(75) Inventors: John Michael Kelly, Drummoyne (AU); Richard Sokolov, Newington (AU); Ian Frederick Johnson, Newington (AU); Ernesto Hueso Monis, Newington (AU); Eric Siu, Newington (AU)

(73) Assignee: ATOMO DIAGNOSTICS PTY LTD, Drummoyne (AU)

(21) Appl. No.: 13/394,330

(22) PCT Filed: Aug. 16, 2010
(86) PCT No.: PCT/AU2010/001048

§ 371 Date: Mar. 5, 2012

(30) Foreign Application Priority Data
Sep. 4, 2009 (AU) 2009904231
Apr. 15, 2010 (AU) 2010901604

(51) Int. Cl. A61B 5/15

(52) U.S. Cl. 600/573

(57) ABSTRACT

A sampling device for collecting a bodily fluid, the device comprising a membrane penetration device including a membrane penetrating element for penetrating a membrane to release a bodily fluid and a collecting element adapted to take up the bodily fluid, the collecting element being engaged with the membrane penetration device.

### TECHNICAL FIELD

The present invention relates generally to devices and methods for sampling or collection of bodily fluids.

### BACKGROUND OF THE DISCLOSURE

There are many medical conditions in humans and animals for which it is desirable to draw a fluid sample for analysis. For example, in diagnosis of various diseases, a blood sample drawn from a patient is analyzed for the presence of a blood-borne pathogen. Alternatively a blood sample can be used to determine the presence or absence of healthy levels of specific blood components. In the case of diabetes, blood samples drawn periodically from a patient are used to monitor blood sugar levels. Blood samples are also screened for molecular diagnostics to provide diagnosis, classification, detection, monitoring, prognosis or other molecular inference.

Samples taken from a patient, animal or organism may be obtained by penetrating the skin of the user using a piercing, slicing, puncturing, pricking, or cutting element such as a lancet device. A lancet device typically includes a body and a lancet. The body is typically adapted to be held by the user, the lancet being coupled to the body and being adapted to pierce the skin of the patient so as to draw blood therefrom. In some lancet devices, the lancet extends from the body at all times. In other lancet devices, the lancet is adapted to be moved, when actuated, from a retracted position in which the lancet tip is disposed within the body to an extended position in which the lancet tip extends beyond the body. Typically, the movement of the lancet from its retracted position to its extended position is effected with a force that means contact of the moving lancet tip with the skin of a patient results in the piercing of the skin of the patient. In many such lancet devices having a movable lancet, the lancet is automatically drawn back into the body after reaching its extended position in order to minimize the risk of inadvertent lancet sticks.

After the skin, typically of a finger, heel or toe, is pricked by the lancet the blood must be expressed from the finger for testing and diagnosis. Testing can be performed using a variety of methods.

In one method, dried blood spot specimens are collected by applying a few drops of expressed blood onto absorbent filter paper manufactured for the task. The blood is allowed to thoroughly saturate the paper and is air dried for several hours. Specimens are stored in low gas-permeability plastic bags with desiccant added to reduce humidity, and may be kept at ambient temperature, even in tropical climates. Once in the laboratory, the dried blood is eluted out in phosphate buffered saline, sometimes overnight. The resultant plate containing the elutes forms the “master” from which dilutions can be made for subsequent testing.

In a second method, blood is expressed into a container such as a micro-tube or similar. The wet blood is then stored or transported in the container for testing.

### SUMMARY OF THE INVENTION

Disclosed is a sampling device for collecting a bodily fluid, the device comprising a membrane penetration device comprising a membrane penetrating element for penetrating a membrane to release a bodily fluid; and, a collecting element adapted to take up the bodily fluid, the collecting element being engaged with the membrane penetration device.

In one form the collecting element comprises a swab.

In one form the swab is a flocked swab.

In one form the membrane penetration device further comprises a sheath having an internal cavity and an opening extending from the cavity wherein the membrane penetration element is moveable between a housed position in which it is disposed substantially inside the sheath and an extended position in which at least a portion of the membrane penetration element extends from the sheath through the opening.

In one form the movement of the membrane penetration element between the housed position and the extended position while the sampling device is positioned proximal to a membrane is operable to penetrate the membrane.

In one form the swab is disposed on the sheath.

In one form the swab is disposed around the opening.

In one form the swab is disposed adjacent the opening.

In one form the swab is disposed such that it covers the opening and the movement of the membrane penetration element between the housed position and the extended position penetrates the swab.

In one form the membrane is skin and the bodily fluid is blood.

In one form the swab is adapted such that contact with the bodily fluid results in take up of that fluid into the swab.

In one form the swab is adapted such that the bodily fluid is released upon contact with a fluid.

In one form the membrane penetration device further comprises an outer housing, the outer housing having a support upon which the sheath is supported, and a cap, the support and cap defining an internal cavity in which the sheath, membrane penetrating element and swab are disposed.

In one form the collecting element is a container.

In one form the container comprises a body defining an interior cavity and a cap, the body being adapted to removably engage with the membrane penetration device such that in a first engaged position the body covers at least a portion of the membrane penetration device.

In one form the body extends from an open end to a closed end and the cap is adapted to be removably engaged with the body such that in a first engaged position the cap is engaged with the closed end of the body.

In one form the cap is adapted to engage with the membrane penetration device.

In one form the cap is adapted to removably cover the open end of the body in a closed position.

In one form the collecting element is removably engaged with the membrane penetration element.

The sampling device allows a user to prepare the site by sterilizing it, collect a bodily fluid from the site of membrane penetration without additional steps such as finding a collection device and expressing the fluid onto the device. In some forms the simplicity is increased by removing the necessity of finding an alcohol wipe to prepare the site and a drying or cleansing wipe for treatment afterwards. This significantly simplifies the process of collecting blood either for a health care professional or for a home user. In one form the sampling device allows a user to sample a bodily fluid and then detach the swab or test strip for pathology. The swab or test strip can be detached by snapping a portion of the sampling device off, removing a portion of the sampling device which has been engaged with the sampling device for sampling, or peeling off or otherwise removing the swab or test strip.

In a second aspect, disclosed is a sampling device for collecting a bodily fluid, the device comprising a membrane penetration device including a membrane penetration element for penetrating a membrane to release a bodily fluid; and, a device engagement member adapted to engage with a test strip or a swab.

In one form the membrane penetration device further comprises a sheath, the sheath having an internal cavity and an opening extending from the cavity, wherein the membrane penetration element is moveable between a housed position in which it is disposed substantially inside the sheath and an extended position in which at least a portion of the membrane penetration element extends from the sheath through the opening.

In one form the device engagement member is disposed on the sheath.

In one form the device engagement member is disposed adjacent or about the opening.

In one form the device further comprises a swab, the swab having a swab engagement' member adapted to engage with the device engagement member and affix the swab to the sampling device.

In one form the device engagement member and the swab engagement member are designed such that the swab is releasably engaged with the device.

In a third aspect disclosed is a sampling device for collecting a bodily fluid, the sampling device comprising a membrane penetration device including a membrane penetration element for penetrating a membrane to release a bodily fluid; and a container for retaining at least a portion of the bodily fluid after release.

In one form the membrane penetration element has a penetration end and the container is in the form of a cap adapted to cover the penetration end of the membrane penetration element element prior to use and retain the bodily fluid after use.

In one form the container comprises a sealing closure.

In one form the sealing closure is adapted to engage with the container in an open configuration and a closed configuration.

In one form the sealing closure is hingedly engaged with the container.

In one form the container extends from an open end to a closed end and the sealing closure is adapted to engage with the closed end while the container is in an open configuration.

In one form the sealing closure is adapted to engage the membrane penetration device when the container is open and to engage the container to close the container.

In a fourth aspect disclosed is a sampling device for collecting a bodily fluid, the device comprising a membrane penetration device including a membrane penetration element for piercing a membrane to release a bodily fluid; and a cap, the cap being adapted to store bodily fluids and including a cap closure element for closing the cap.

In one form the cap comprises a container body, adapted to removably cover the membrane penetration element when the cap is in an open configuration, and a container lid adapted to seal the container body when the cap is in a closed configuration.

In one form the container lid is adapted to engage with the container body in both the closed configuration and the open configuration.

In one form the container lid is hingedly connected with the container body.

In one form the container body extends from a closed end to an open end and the container lid is adapted to engage with the closed end when the container body is in an open configuration and the open end when the container body is in a closed configuration.

In one form the sealing closure is adapted to engage the sampling device when the container is in an open configuration and to engage the container to close the container when the container is in a closed configuration.

The device allows a user to collect a bodily fluid from the site of membrane penetration without additional steps such as finding a container. This simplifies the process of collecting blood either for a health care professional or for a home user.

In use, a user removes the container from its position capping the sampling device, pierces the skin with the membrane penetration element, expresses bodily fluid into the container and closes the container with the sealing cover. The container is then sealed and, if necessary, can be transported for testing.

### BRIEF DESCRIPTION OF THE DRAWINGS

A preferred embodiment will now be described by way of example only, with reference to the accompanying drawings in which:

FIG. 1 is a cross sectional view of the sampling device of one embodiment of the present invention;

FIG. 2 is a cross sectional view of the sampling device of one embodiment of the present invention;

FIG. 3 is a cross sectional view of the sampling device of one embodiment of the present invention;

FIG. 4 is a cross sectional view of the sampling device of one embodiment of the present invention;

FIG. 5 is a cross sectional view of the sampling device of one embodiment of the present invention;

FIG. 6 is a cross sectional view of the sampling device of one embodiment of the present invention;

FIG. 7 is a cross sectional view of the sampling device of one embodiment of the present invention;

FIG. 8 is a cross sectional view of the sampling device of one embodiment of the present invention;

FIG. 9 is a cross sectional view of the sampling device of one embodiment of the present invention;

FIG. 10 is a perspective view of one embodiment of the present invention;

FIG. 11 is an exploded perspective view of the embodiment of FIG. 10;

FIG. 12 is a side view of the embodiment of FIG. 10;

FIG. 13 is a side view of the embodiment of FIG. 10 in use;

FIG. 14 is a perspective view of the embodiment of FIG. 10 in use;

FIG. 15 is a perspective view of the embodiment of FIG. 10 in use;

FIG. 16 is a perspective view of the embodiment of FIG. 10 in use;

FIG. 17 is a perspective view of the embodiment of FIG. 10 in use;

FIG. 18 is a perspective view of the embodiment of FIG. 10 in use;

FIG. 19 is a perspective view of the embodiment of FIG. 10 in use;

FIG. 20A is a perspective view of a portion of the embodiment of FIG. 10 in use;

FIG. 20B is a perspective view of a portion of the embodiment of FIG. 10 in use;

FIG. 21 is a side view of one embodiment of the present invention;

FIG. 22 is a perspective view of the embodiment of FIG. 21;

FIG. 23 is a front view of the embodiment of FIG. 21;

FIG. 24 is a side view of the embodiment of FIG. 21;

FIG. 25 is a front view of the embodiment of FIG. 21;

FIG. 26 is a top view of the embodiment of FIG. 21;

FIG. 27 is a perspective view of the embodiment of FIG. 21;

FIG. 28 is a side view of the cap of the embodiment of FIG. 21;

FIG. 29 is a perspective view of the cap of the embodiment of FIG. 21;

FIG. 30 is a perspective view of one embodiment of the present invention;

FIG. 31 is a perspective view of the embodiment of FIG. 30;

FIG. 32 is a top view of the embodiment of FIG. 30;

FIG. 33 is a front view of the embodiment of FIG. 30;

FIG. 34 is a side view of the embodiment of FIG. 30;

FIG. 35 is a side view of the cap of the embodiment of FIG. 30;

FIG. 36 is a perspective view of the cap of the embodiment of FIG. 30;

FIG. 37 is a perspective view of the cap of the embodiment of FIG. 30;

FIG. 38 is a perspective view of the cap of the embodiment of FIG. 30;

FIG. 39 is a perspective view of the cap of the embodiment of FIG. 30;

FIG. 40 is a side view of the device of a further embodiment of the present invention;

FIG. 41 is a front view of the embodiment of FIG. 40;

FIG. 42 is a perspective view of the embodiment of FIG. 40.

### DETAILED DESCRIPTION OF EMBODIMENTS

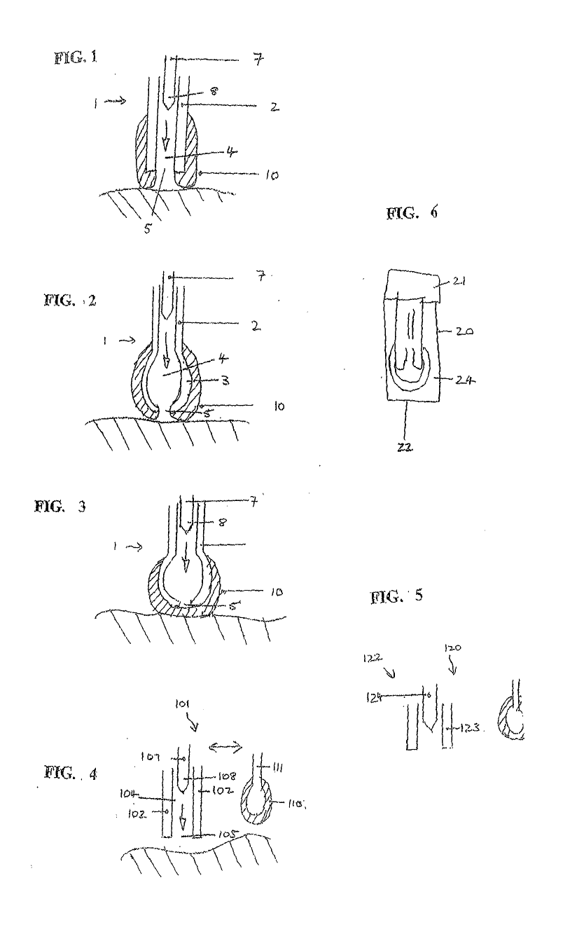

In a first embodiment as shown in FIG. 1 and FIG. 6, disclosed is a sampling device 1 for collecting a sample of a bodily fluid. The sampling device is described with reference to collecting blood samples, though a person skilled in the art will be aware that other bodily fluids may be collected using the sampling device.

The sampling device 1 comprises a sheath 2 defining an internal cavity 4. The sheath 2 includes an opening 5 that extends from the internal cavity 4. A lancet 7 such as a needle or blade or other sharp is disposed prior to use in the internal cavity 4. The lancet 7 is moveable from a housed position in which the lancet 7 is substantially disposed within the sheath 2. In this position the lancet is protected by the sheath 2. This is shown in FIG. 1. The lancet is moveable to an extended position (not illustrated) in which the lancet 7 extends at least partly from the sheath 2. In this position the tip 8 of the lancet 7 is positioned outside the sheath 2 such that a user can prick the skin with the tip 8 of the lancet 7.

The movement of the lancet 7 from the housed position to the extended position is performed with sufficient force to pierce the skin of a user such that if the sheath 2 is positioned against the skin of the user and movement of the lancet between the housed and the extended position is actuated the skin will be pierced by the lancet.

The sampling device 1 further comprises a swab 10 which is disposed around the opening 5. The swab 10 comprises an absorbent material however in a preferred form the swab comprises an absorbent material which is adapted to allow the sample to be released upon soaking or immersing the swab in a fluid. A flocked swab material is advantageous for this application as it provides for great absorbency as well as sample release.

The swab 10 is disposed about the opening 5 such that when the lancet 7 pierces the skin, the expressed blood resulting from the piercing is easily absorbed by moving the swab in contact with the fluid.

The sampling device 1 further includes an outer housing 20. The outer housing 20 is made up of a support 21 upon which the sheath 2 is supported. The outer housing 20 further includes a cap 22 which is adapted to define a cavity 24 in which the sheath and swab are located prior to use.

In use, a user removes the cap 22 from the outer housing 20. The user then grasps the support 21 and locates the swab 10 against the skin at the position where the blood is to be sampled. The user actuates the device such that the lancet 7 moves from the housed position to the extended position, piercing the skin and allowing blood to be expressed. The lancet then returns automatically to the housed position.

The blood which is expressed from the piercing is then taken up by the swab 10 merely by contacting the blood with the swab 10. A particular amount of blood can be stored by the swab depending upon the size of the swab. The area of the swab controls the volume of blood stored making the device suitable with quantitative diagnostic applications. The user then replaces the cap 22 on the outer housing 20 such that the sampling device can be transported for diagnostics.

A diagnostician will then remove the cap 22 and place the swab in a fluid such as an assay, reagent, buffer solution or transport medium to release the sample.

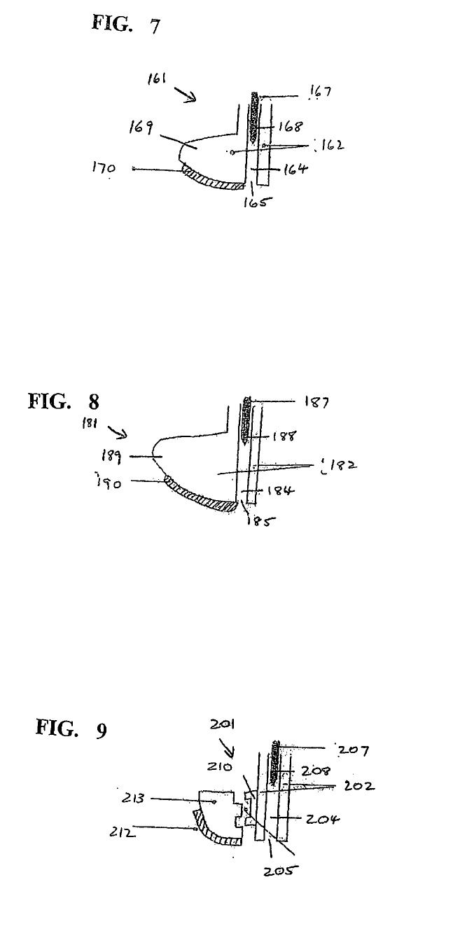

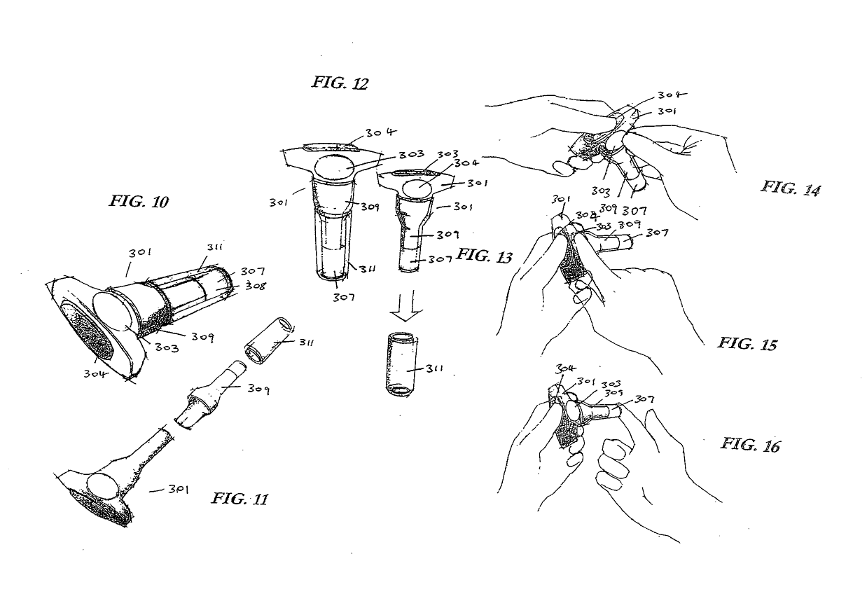

In a second embodiment, shown in FIG. 2, the sheath 2 includes a curved section 3 which is positioned proximal to the opening 5. The curved section 3 provides a support for the swab 10 which allows the overall sampling device to simulate the appearance of an ordinary cotton swab. This effect can also be performed by thickening the material of the sheath 2 in a section proximal to the opening 5 or by thickening the swab 10.

In a third embodiment, shown in FIG. 3, the swab 10 extends over the opening 5. In this embodiment the movement of the lancet 7 between the housed position shown in FIG. 3 and the extended position in which the tip 8 of the lancet 7 extends out of the sheath 2 pierces the swab 10 and the skin. This embodiment allows the sampling device 1 to simulate the appearance of a cotton swab more convincingly as the lancet 7 can not be seen before the sampling device 1 is used.

In a fourth embodiment shown in FIG. 4, the sampling device 101 comprises a sheath 102 defining an internal cavity 104. The sheath 102 includes an opening 105 that extends from the internal cavity 104. A lancet or sharp 107 such as a needle or blade is disposed in the internal cavity 104. The lancet 107 is moveable from a housed position in which the lancet 107 is substantially disposed within the sheath 102. In this position the lancet is protected by the sheath 102, as shown in FIG. 4. The lancet 107 is moveable to an extended position (not illustrated) in which the lancet 107 extends at least partly from the sheath 102. In this position the tip 108 of the lancet 107 is positioned outside the sheath 102 such that a user can prick the skin with the tip 108 of the lancet 107.

The movement of the lancet 107 from the housed position to the extended position is performed with sufficient force to pierce the skin of a user.

The sampling device 101 further comprises a swab 110 which is positioned on a support 111. The swab 110 comprises an absorbent material however in a preferred form the swab comprises an absorbent material which is adapted to allow the sample to be released upon soaking the swab in a fluid. A flocked swab material is advantageous for this application as it provides for great absorbency as well as sample release.

The swab 110 is connected with the sheath by means of a fixed or flexible attachment method or alternatively the sheath includes an opening end 105 and a swab end (not illustrated).

In a fifth embodiment, the sampling device comprises a sampling kit 120 which is made up of a piercing device 122 and a swab 130. The piercing device 122 comprises a sheath 123 and a lancet 124 disposed in the sheath.

In yet another embodiment, the sampling device is designed such that after sampling blood, the swab 10 and a portion of the sheath 2 can be broken off and sent for diagnostics. In this not illustrated embodiment a rupture line is created in the sheath so that a portion of the sheath can be removed. This means the swab 10 and a portion of the sheath 2 can be sent to diagnostics without the lancet causing a pathologist or technician danger. The portion of sheath and swab are sized to be mailed.

In another embodiment, the sampling device comprises a sheath defining an internal cavity. The sheath includes an opening 5 that extends from the internal cavity. A lancet or sharp such as a needle or blade is disposed in the internal cavity. The lancet is moveable from a housed position in which the lancet is substantially disposed within the sheath. In this position the lancet is protected by the sheath. This is shown in FIG. 1. The lancet is moveable to an extended position in which the lancet extends at least partly from the sheath. In this position the tip of the lancet is positioned outside the sheath 2 such that a user can prick the skin with the tip of the lancet.

In a further embodiment shown in FIG. 7, the sampling device 161 comprises a sheath 162 defining an internal cavity 164. The sheath 162 includes an opening 165 that extends from the internal cavity 164. A lancet or sharp 167 such as a needle or blade is disposed in the internal cavity 164. The lancet 167 is moveable from a housed position in which the lancet 167 is substantially disposed within the sheath 162. In this position the lancet is protected by the sheath 162. The lancet 167 is moveable to an extended position (not illustrated) in which the lancet 167 extends at least partly from the sheath 162. In this position the tip 168 of the lancet 167 is positioned outside the sheath 162 such that a user can prick the skin with the tip 168 of the lancet 167.

The sheath 162 includes a projection 169 which extends outwardly from the sheath 162. A swab 170 is positioned adjacent the opening 165 on the projection 169 and extending outwardly from the sheath 162. In this position a user can pierce the skin, determine that sufficient blood or other bodily fluid has been expressed, and roll the sampling device 161 so that the swab 170 contacts the bodily fluid and absorbs it for sampling.

In a further embodiment shown in FIG. 8, the sampling device 181 comprises a sheath 182 defining an internal cavity 184. The sheath 182 includes an opening 185 that extends from the internal cavity 184. A lancet or sharp 187 such as a needle or blade is disposed in the internal cavity 184. The lancet 187 is moveable from a housed position in which the lancet 187 is substantially disposed within the sheath 182. In this position the lancet is protected by the sheath 182. The lancet 187 is moveable to an extended position (not illustrated) in which the lancet 187 extends at least partly from the sheath 182. In this position the tip 188 of the lancet 187 is positioned outside the sheath 182 such that a user can prick the skin with the tip 188 of the lancet 187.

The sheath 182 includes a projection 189 which extends outwardly from the sheath 182. A test strip 190 is positioned adjacent the opening 185 on the projection 189 and extending outwardly from the sheath 182. In this position a user can pierce the skin, determine that sufficient blood or other bodily fluid has been expressed, and roll the sampling device 181 so that the test strip 190 contacts the bodily fluid to make a diagnosis or reading.

In a further embodiment shown in FIG. 9, the sampling device 201 comprises a sheath 202 defining an internal cavity 204. The sheath 202 includes an opening 205 that extends from the internal cavity 204. A lancet or sharp 207 such as a needle or blade is disposed in the internal cavity 204. The lancet 207 is moveable from a housed position in which the lancet 207 is substantially disposed within the sheath 202. In this position the lancet is protected by the sheath 202. The lancet 207 is moveable to an extended position (not illustrated) in which the lancet 207 extends at least partly from the sheath 202. In this position the tip 208 of the lancet 207 is positioned outside the sheath 202 such that a user can prick the skin with the tip 208 of the lancet 207.

An engagement member 210 is positioned adjacent the opening 205 and extending outwardly from the sheath 202. The engagement member is adapted such that a test strip or swab 212 with a corresponding engagement member 213 can be engaged with the sampling device 201 in order to allow easy sampling or testing. The test strip or swab 212 can be engaged by a health professional or forwarded to the user for self-sampling. A user can pierce the skin, determine that sufficient blood or other bodily fluid has been expressed, and roll the sampling device 201 so that the test strip or swab 212 contacts the bodily fluid to make a sampling, diagnosis or reading.

The engagement member 190 and 210 comprises a clip, fastener or snap-fit or any other means of engagement that allows the swab or test strip to be easily and removably affixed to the sampling device.

The test strip is adapted to receive the sample and either provide a readable result for a home diagnosis or be forwarded for diagnostics.

FIGS. 10 through 20 show an embodiment of the present invention in an operation sequence. In this embodiment the lancet body 301 is substantially T-shaped. The lancet body 301 includes an alcohol wipe pad 303 and a push button 304. A swab 307 including a flocked area 308 is attached with a swab body 309 to engage with the lancet body 301. A cap 311 is adapted to cover the swab 307.

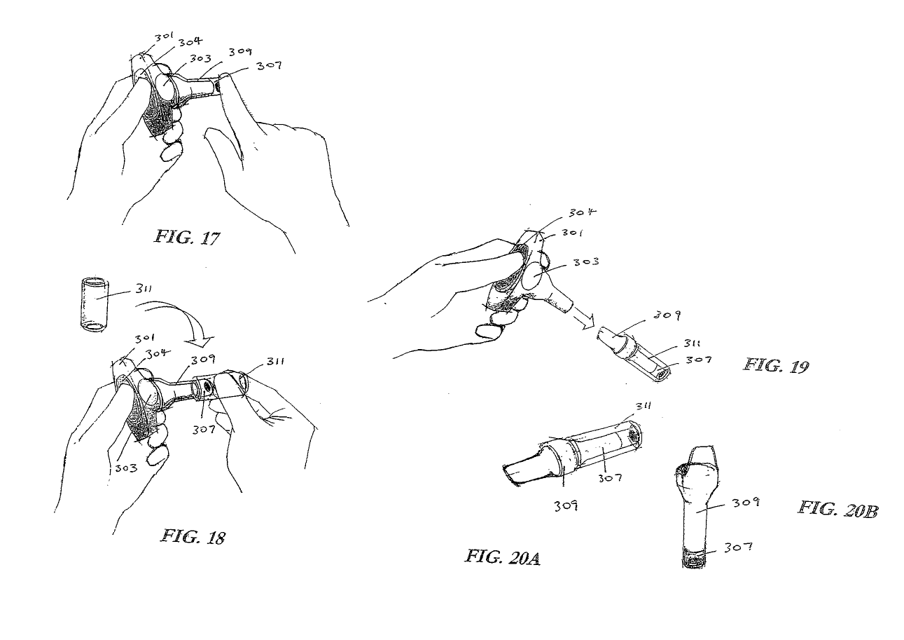

A user proceeds to remove the cap 311 from the lancet body 301 to expose the swab 307. The user then utilizes the alcohol wipe pad 303 to clean the site for piercing as shown in FIGS. 14 and 15. The user wipes away the first drop of fluid at the piercing site using the sterile dry pad. As shown in FIG. 16, the user then lightly presses the site for piercing (in this case a finger) against the swab 307 and hence in line with the lancet tip in the lancet body 301. The lancet is activated using the push button 304. As shown in FIG. 17, blood is then expressed onto the flocked portion 308 of the swab 307. The cap 311 is replaced to contain the blood sample from contamination. The swab body 309 and swab 307 along with the cap can then be removed from the overall device to be transported for testing as shown in FIG. 19.

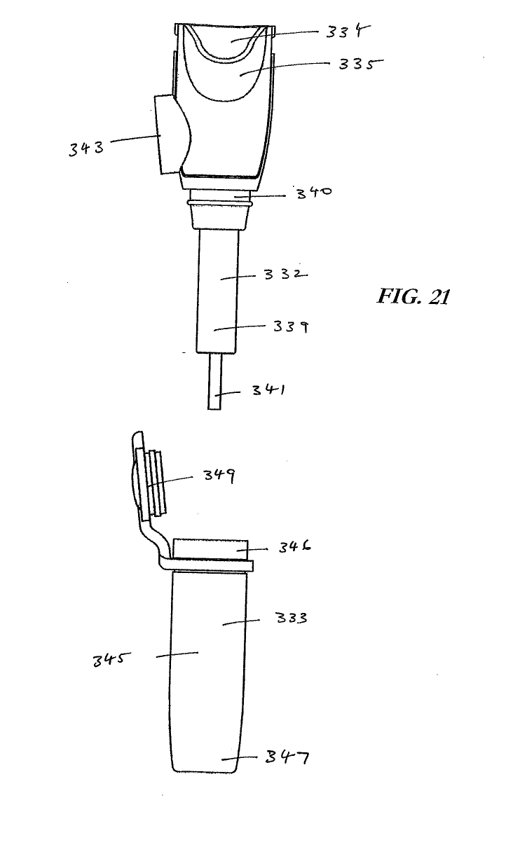

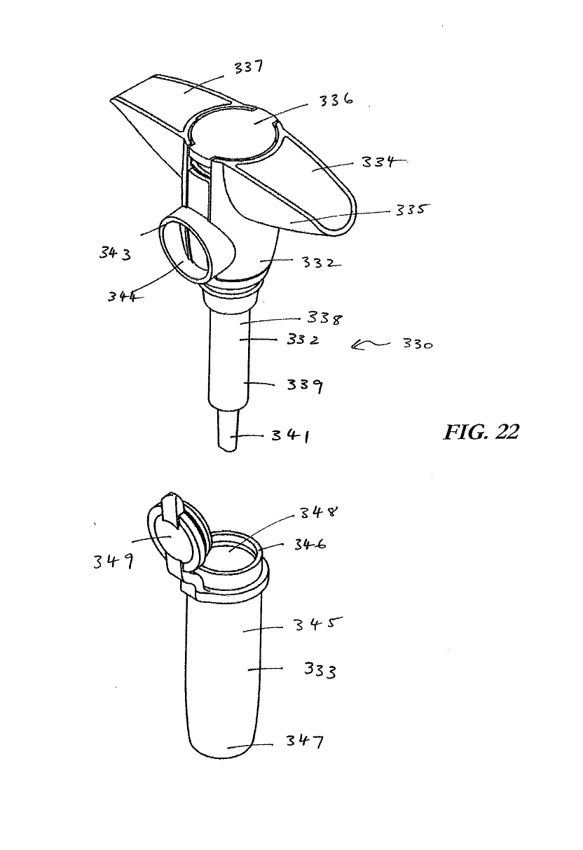

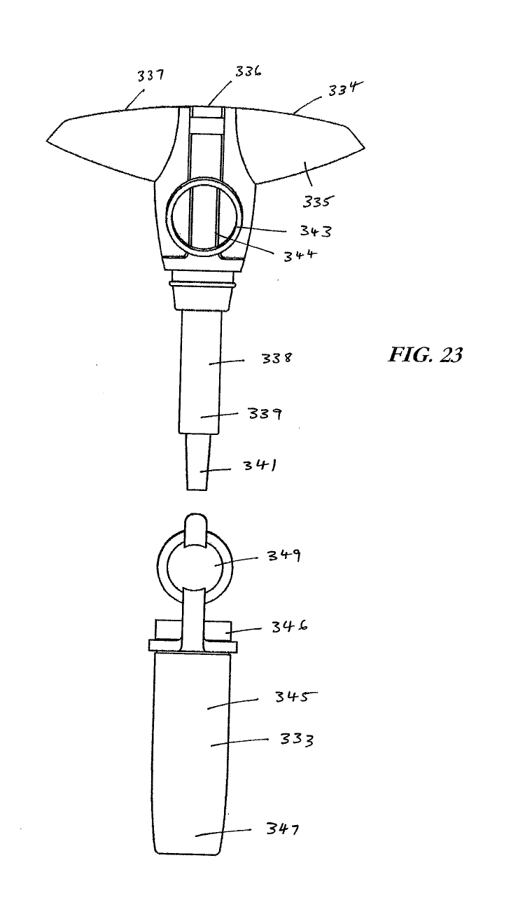

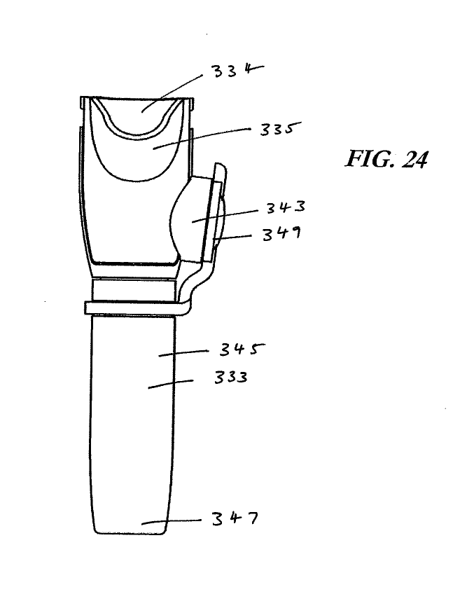

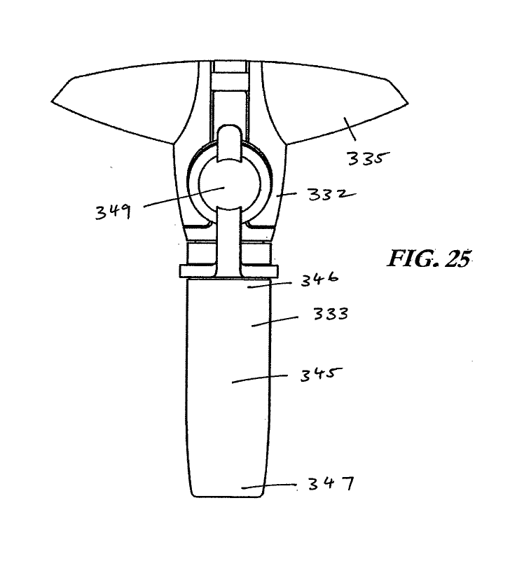

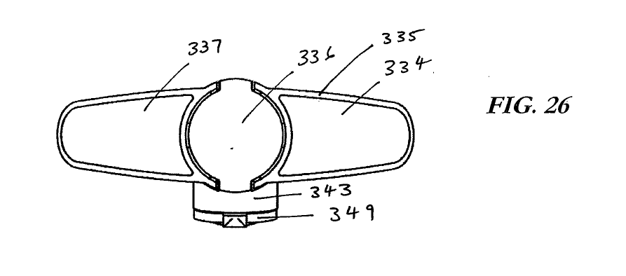

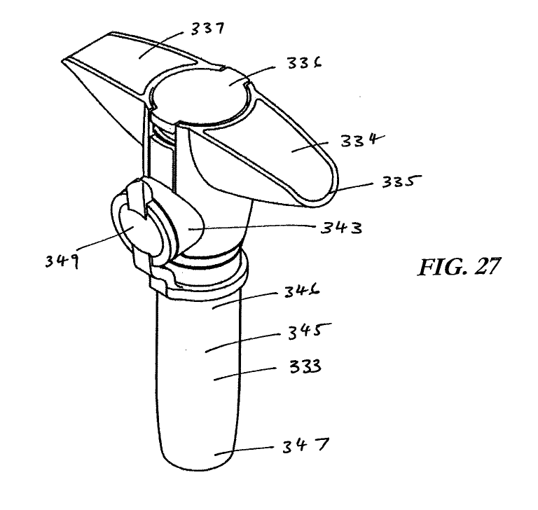

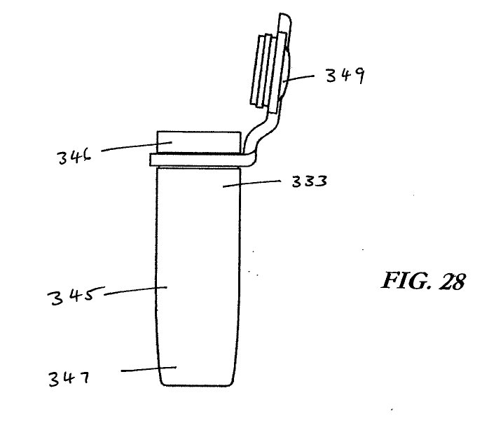

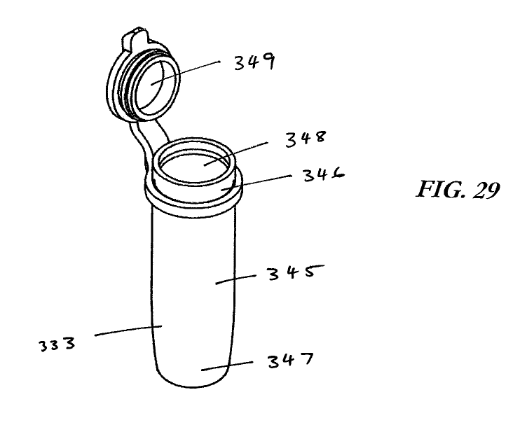

FIGS. 21 through 29 show a further aspect of the present application. In this aspect, disclosed is a sampling device 330 comprising a lancet body 332 and a container 333. The lancet body 332 is substantially T-shaped and comprises a piercing element (internal) which is moveable between a retracted position in which the piercing element is positioned within the lancet body 332 and an extended position in which the lancet extends beyond the lancet body 332 to allow for piercing of skin or a membrane for release of bodily fluids. The bar portion 335 of the lancet body 332 is adapted to be held by a user's hand. The bar portion 335 includes a push button 336 for activation of the piercing element between the retracted position and the extended position. In one form, the bar portion 335 further includes an alcohol swab locator 334 where and alcohol swab is sealed into position under a removable sealing cover on one side of the bar portion 335. In one form the bar portion 335 further includes a cleaning wipe locator 337 where a wipe is located under a removable sealing cover.

The stem portion 338 of the lancet body 332 comprises a barrel 339 and a shoulder 340 along with a lancet tip which is positioned behind overmolded protective cover 341. The lancet tip is exposed when the overmolded tip 341 is twisted off. The stem portion 338 further includes a closure receiving portion 343 which includes a cavity 344.

The device further comprises a container 333 which is adapted to fit over the stem portion 338 of the lancet body 332 when the device is not in use. The container 333 comprises a vessel body 345 extending from an open end 346 to a closed end 347. The open end 346 includes an opening 348. A sealing closure 349 is hingedly affixed with the vessel body 345 and is adapted to seal the opening 348.

Prior to use, the container 333 acts as a cap to the lancet body 332, covering the lancet tip 341 and barrel 339 and abutting a portion of the shoulder 340. The sealing closure 349 engages the closure receiving portion 343 at cavity 344. In use a user removes the closure 349 and the container 333 from the lancet body 332. The user then utilizes the device to pierce skin. Blood or other bodily fluid is then expressed into the container 333. The user then closes the container 333 using the sealing closure 349, thus preparing the container for transportation.

This device is designed for sampling wet blood and sealing and storing it for transportation. The container may, in one form, include indicia for indicating the volume of fluid held in the container 333. The container may also include space for labeling the container 333 for transport and identification. The container may also include a scoop or other feature on the rim to aid in the collection of fluid into the container.

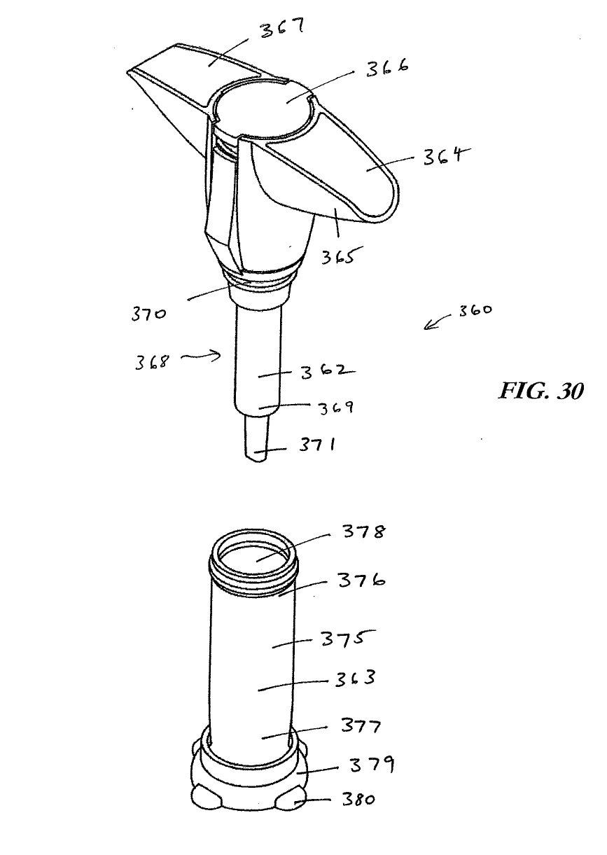

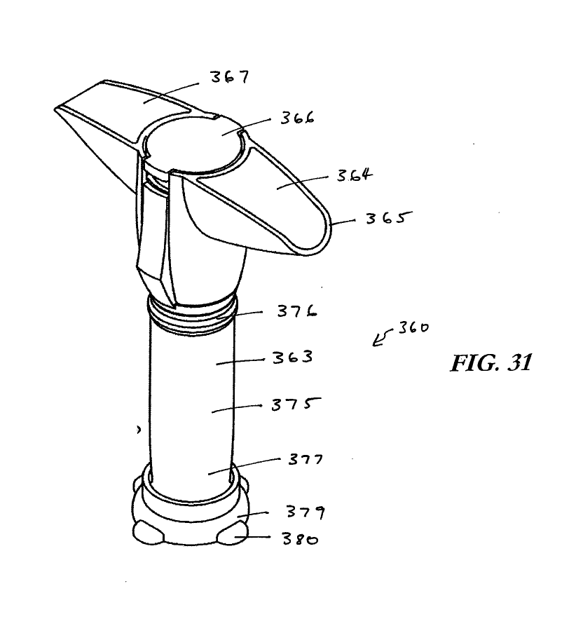

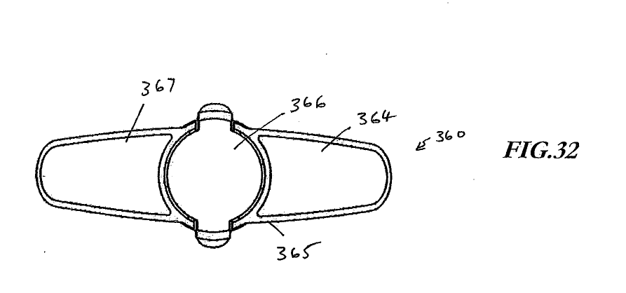

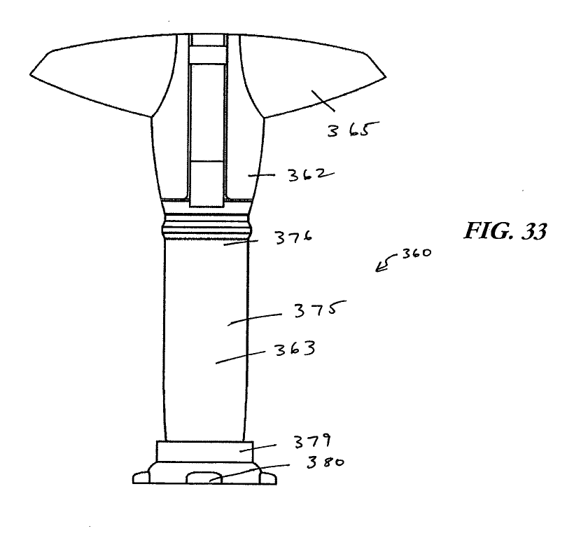

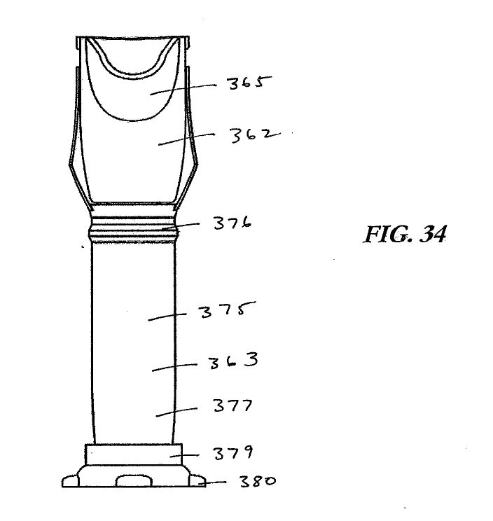

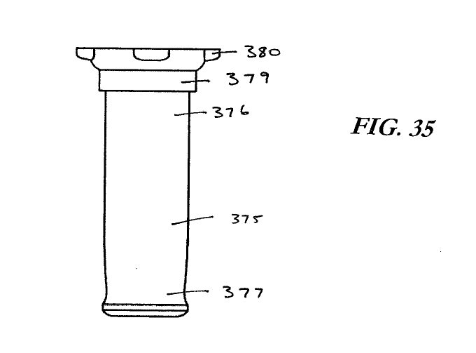

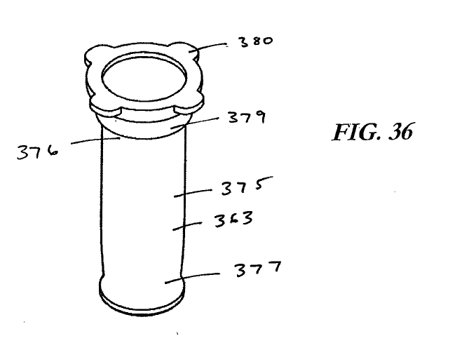

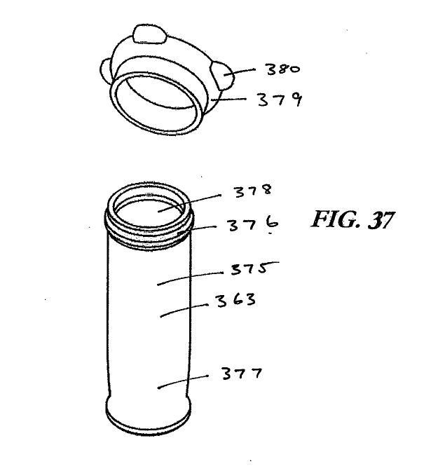

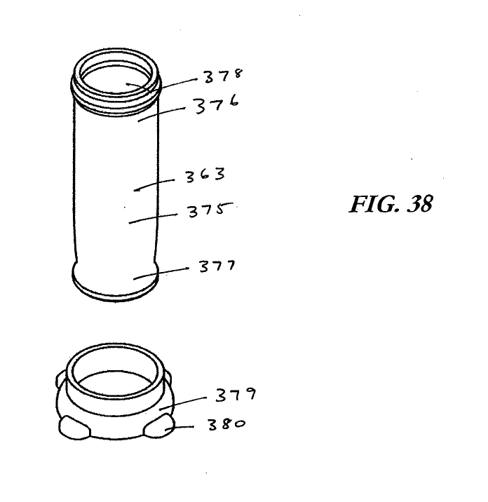

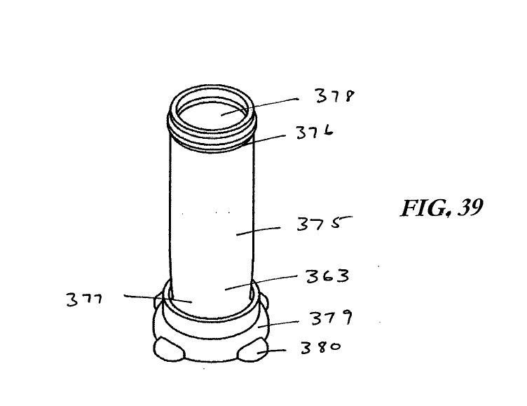

FIGS. 30 to 39 illustrate a further embodiment of the device. Disclosed is a sampling device 360 comprising a lancet body 362 and a container 363. The lancet body 362 is T-shaped and comprises a lancet (internal) which is moveable between a retracted position in which the lancet is positioned within the lancet body 362 and an extended position in which the lancet extends beyond the lancet body 362 to allow for piercing of skin or a membrane for release of bodily fluids.

The bar portion 365 of the lancet body 362 is adapted to be held by a user's hand and includes a push button 366 for activation of the lancet between the retracted position and the extended position. The stem portion 368 of the lancet body 362 comprises a barrel 369 and a shoulder 370 along with a lancet tip 371. In one form, the bar portion 365 further includes an alcohol swab locator 364 where and alcohol swab is sealed into position under a removable sealing cover on one side of the bar portion 365. In one form the bar portion 365 further includes a cleaning wipe locator 367 where a wipe is located under a removable sealing cover.

The device further comprises a container 363 which is adapted to fit over the stem portion 368 of the lancet body 362 when the device is not in use. The container 363 comprises a vessel body 375 extending from an open end 376 to a closed end 377. The open end 376 includes an opening 378. A sealing closure 379 is adapted to be affixed with the vessel body 375 at the closed end 377 by means of an interference fit, a push fit, a threaded connection or any other engagement. The sealing closure 379 is also adapted to engage with the open end 376 to seal the opening 378 by means of a helical thread or any other sealing fit that is resistant to fluid within the container escaping through the seal.

Prior to use, the container 363 acts as a cap to the lancet body 362, covering the lancet tip 371 and barrel 369 and abutting a portion of the shoulder 370. The sealing closure 379 engages the container 363 at closed end 377. In use a user removes the container 363 from the lancet body 332. The sealing closure 379 is affixed with the container 363 and acts to stabilize the container in a standing position such that the open end 376 is oriented for deposit of fluid. The sealing closure 379 includes stabilizing feet 380

The user then utilizes the device to pierce skin. Blood or other bodily fluid is then expressed into the container 363. The user then removes the sealing closure 379 from the closed end 377 of the container 363 and uses the sealing closure to close the container 363, thus preparing the container for transportation.

This device is designed for sampling wet blood and sealing and storing it for transportation.

The container may, in one form, include indicia for indicating the volume of fluid held in the container 363. The container may also include space for labeling the container 363 for transport and identification.

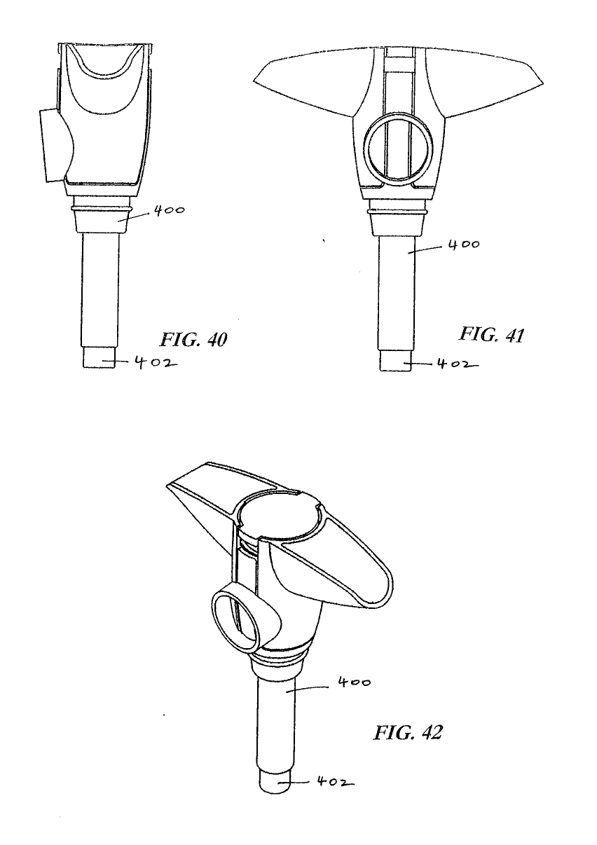

In one form shown in FIGS. 40-42, actuation of the lancet is effected without a push button. In this form, the lancet body 400 includes an actuation tip 402 which is moveable between a rest position and an actuation position. The actuation position is further retracted into the lancet body than is the rest position. Thus in use, a user presses the actuation tip 402 against skin, actuating the lancet.

Throughout the detailed description, the membrane penetrating element has been referred to and described in respect of a lancet as illustrated, however persons skilled in the art will be aware that the lancet could be any piercing, slicing, cutting, puncturing or pricking element which allows a user to penetrate a membrane such as the skin to allow a fluid sample to be released.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

Variations and modifications may be made to the parts previously described without departing from the spirit or ambit of the invention.

1.-39. (canceled)

40. A sampling device for collecting a bodily fluid, the device comprising:
a membrane penetration device comprising a membrane penetration element for penetrating a membrane to release a bodily fluid;
a collecting element adapted to take up the bodily fluid, the collecting element being engaged with the membrane penetration device.

41. A sampling device as defined in claim 40, wherein the collecting element comprises a swab; optionally wherein the swab is a flocked swab.

42. A sampling device as defined in claim 40, wherein the membrane penetration device further comprises a sheath, the sheath having an internal cavity and an opening extending from the cavity wherein the membrane penetration element is moveable between a housed position in which it is disposed substantially inside the sheath and an extended position in which at least a portion of the membrane penetration element extends from the sheath through the opening.

43. A sampling device as defined in claim 42, wherein the movement of the membrane penetration element between the housed position and the extended position while the sampling device is positioned proximal to a membrane is operable to penetrate the membrane.

44. A sampling device as defined in claim 42, wherein the swab is disposed on the sheath; optionally wherein the swab is disposed around the opening; optionally wherein the swab is disposed adjacent the opening; optionally wherein the swab is disposed such that it covers the opening and the movement of the membrane penetration element between the housed position and the extended position penetrates the swab.

45. A sampling device as defined claim 40, wherein the membrane is skin and the bodily fluid is blood; optionally wherein the swab is adapted such that contact with the bodily fluid results in take up of that fluid into the swab; optionally wherein the swab is adapted such that the bodily fluid is released upon contact with a fluid.

46. A sampling device as defined in claim 41, wherein the membrane penetration device further comprises an outer housing, the outer housing having a support upon which the sheath is supported, and a cap, the support and cap defining an internal cavity in which the sheath, membrane penetrating element and swab are disposed.

47. A sampling device as defined in claim 40, wherein the collecting element is a container; optionally wherein the container comprises a body defining an interior cavity and a cap, the body being adapted to removably engage with the membrane penetration device such that in a first engaged position the body covers at least a portion of the membrane penetration device.

48. A sampling device as defined in claim 47, wherein the body extends from an open end to a closed end and the cap is adapted to be removably engaged with the body such that in a first engaged position the cap is engaged with the closed end of the body.

49. A sampling device as defined in claim 47, wherein the cap is adapted to engage with the membrane penetration device.

50. A sampling device as defined in claim 47, wherein the cap is adapted to removably cover the open end of the body in a closed position; optionally wherein the collecting element is removably engaged with the membrane penetration element.

51. A sampling device for collecting a bodily fluid, the device comprising:
a membrane penetration device including a membrane penetration element for penetrating a membrane to release a bodily fluid; and a device engagement member adapted to engage with a test strip or a swab.

52. A sampling device as defined in claim 51, the membrane penetration device further comprising a sheath, the sheath having an internal cavity and an opening extending from the cavity, wherein the membrane penetration element is moveable between a housed position in which it is disposed substantially inside the sheath and an extended position in which at least a portion of the membrane penetration element extends from the sheath through the opening;
optionally wherein the movement of the membrane penetration element between the housed position and the extended position when the sampling device is proximal to a membrane is operable to penetrate the membrane.

53. A sampling device as defined in claim 52, wherein the device engagement member is disposed on the sheath; optionally wherein the device engagement member is disposed adjacent or about the opening.

54. A sampling device as defined in claim 51, further comprising a swab, the swab having a swab engagement member adapted to engage with the device engagement member and affix the swab to the sampling device; optionally wherein the device engagement member and the swab engagement member are designed such that the swab is releasably engaged with the device.

55. A sampling device for collecting a bodily fluid, the sampling device comprising a membrane penetration device including a membrane penetration element for penetrating a membrane to release a bodily fluid; and a container for retaining at least a portion of the bodily fluid after release; optionally wherein the membrane penetration element has a penetration end and the container is in the form of a cap adapted to cover the penetration end of the membrane penetration element prior to use and retain the bodily fluid after use,

56. A sampling device as defined in claim 55, wherein the container comprises a sealing closure; preferably wherein the sealing closure is adapted to engage with the container in an open configuration and a closed configuration; preferably wherein the sealing closure is hingedly engaged with the container.

57. A sampling device as defined in claim 56, wherein the container extends from an open end to a closed end and the sealing closure is adapted to engage with the closed end while the container is in an open configuration; optionally wherein the sealing closure is adapted to engage the membrane penetration device when the container is open and to engage the container to close the container.

58. A sampling device for collecting a bodily fluid, the device comprising a membrane penetration device including a membrane penetration element for piercing a membrane to release a bodily fluid; and a cap, the cap being adapted to store bodily fluids and including a cap closure element for closing the cap.

59. A sampling device as defined in claim 58, wherein the cap comprises a container body, adapted to removably cover the membrane penetration element when the cap is in an open configuration, and a container lid adapted to seal the container body when the cap is in a closed configuration; wherein the container lid is adapted to engage with the container body in both the closed configuration and the open configuration; optionally wherein the container lid is hingedly connected with the container body; optionally wherein the container body extends from a closed end to an open end and the container lid is adapted to engage with the closed end when the container body is in an open configuration and the open end when the container body is in a closed configuration; optionally wherein the sealing closure is adapted to engage the sampling device when the container is in an open configuration and to engage the container to close the container when the container is in a closed configuration.
